(19) United States
(12) Patent Application Publication
(10) Pub. No.: US 2017/0332993 A1
(43) Pub. Date: Nov. 23, 2017

(54) WHEEZING-RELATED INFORMATION DISPLAY APPARATUS

(71) Applicant: OMRON HEALTHCARE CO., LTD., Muko-shi (JP)

(72) Inventor: Kei ASAI, Kyoto (JP)

(73) Assignee: OMRON HEALTHCARE CO., LTD., Muko-shi (JP)

(21) Appl. No.: 15/669,557

(22) Filed: Aug. 4, 2017

Related U.S. Application Data
(63) Continuation of application No. PCT/JP2015/082505, filed on Nov. 19, 2015.

(30) Foreign Application Priority Data
Feb. 27, 2015 (JP) 2015-039258

(51) Int. Cl. A61B 7/00; A61B 7/04; A61B 5/08; A61B 5/00

(52) U.S. Cl. CPC A61B 7/003; A61B 5/742; A61B 7/04; A61B 5/0803; A61B 5/7275; A61B 5/6833; A61B 2503/04

(57) ABSTRACT

A wheezing-related information display apparatus includes a breathing sound detection unit that detects a breathing sound of a measurement subject and acquires a breathing signal in a time series indicating the breathing sound. The wheezing-related information display apparatus includes a determination processing unit that, based on the breathing sound signal, determines whether or not wheezing is included in the breathing sound in each predetermined processing unit period. The wheezing-related information display apparatus includes a display processing unit that displays information indicating temporal change in the frequency of wheezing on a display screen based on the result of the determination performed by the determination display unit.

### TECHNICAL FIELD

The present invention relates to a wheezing-related information display apparatus, and more specifically relates to a wheezing-related information display apparatus that displays information related to wheezing included in a breathing sound of a measurement subject.

### BACKGROUND ART

For example, Patent Document 1 (US 2011/0125044 A1) discloses an automated system for observing a respiratory disease such as asthma. The system provides a summary of data and a warning when the severity of symptoms reaches a threshold value based on data from a microphone and an accelerometer. In particular, for wheezing, peaks of a frequency spectrum in a frequency range of about 200 to 800 Hz are measured, the peaks of the frequency spectrum and a predetermined value that is associated with wheezing and stored in the memory are compared, and the result of the comparison is used as an element for determining the severity.

### CITATION LIST

### Patent Literature

Patent Document 1: US 2011/0125044 A1

### SUMMARY OF INVENTION

### Technical Problem

Incidentally, it is well known that in the case of a doctor diagnosing whether or not a patient has asthma, temporal change in the frequency of wheezing is one diagnostic factor.

However, Patent Document 1 (US 2011/0125044 A1) does not disclose display of temporal change in the frequency of wheezing.

In view of this, the present invention aims to provide a wheezing-related information display apparatus that can display temporal change in wheezing included in a breathing sound of a measurement subject.

### Solution to the Problem

In order to solve the above-described problem, the wheezing-related information display apparatus of the present invention includes:

a breathing sound detection unit configured to detect a breathing sound of a measurement subject and acquire a breathing sound signal in a time series expressing the breathing sound;

a determination processing unit configured to, based on the breathing sound signal, for each predetermined processing unit period, determine whether or not wheezing is included in the breathing sound; and

a display processing unit configured to, based on a result of determination performed by the determination processing unit, display information indicating temporal change in a frequency of the wheezing on a display screen.

Here, the “processing unit period” is typically set to around an amount of time needed by the determination processing unit for calculation processing. For example, various amounts of time, such as 0.05 seconds or 0.1 second, can be set according to the calculation capability of the determination processing unit.

Here, in the wheezing-related information display apparatus of the present invention, the breathing sound detection unit detects the breathing sound of the measurement subject and acquires the breathing sound signal in a time series indicating the breathing sound. Based on the breathing sound signal, for each predetermined processing unit period, the determination processing unit determines whether or not wheezing is included in the breathing sound. Based on the result of the determination performed by the determination processing unit, the display processing unit displays information indicating the temporal change in the frequency of the wheezing on the display screen. Accordingly, the user (typically indicates the measurement subject, a guardian or caregiver who cares for the measurement subject, a medical professional such as a nurse, or the like) can be made aware of the temporal change in the frequency of the wheezing included in the breathing sound of the breathing subject. If the temporal change in the frequency of the wheezing is known in real time, it is possible to know whether or not the symptoms of asthma are worsening, and therefore a preventative measure such as administration of medication can be taken, which leads to prevention of the worsening of asthma. Also, if the information indicating the temporal change in the frequency of the wheezing is stored in the storage unit (e.g., a memory or the like), the user can show the temporal change in the frequency of the wheezing included in the breathing sound of the measurement subject to a doctor by causing the information to be read out and displayed on the display screen the next time the measurement subject has a medical examination. As a result, it is easier for the doctor to diagnose whether or not the measurement subject has asthma and the severity of the asthma, and the doctor can easily create a treatment plan.

With the wheezing-related information display apparatus according to an embodiment, as information indicating the temporal change in the frequency of the wheezing, the display processing unit displays a frequency of processing unit periods in which it was determined by the determination processing unit that wheezing is included, in each addition unit period including a plurality of the processing unit periods.

Here, the “addition unit period” can be set to various periods, such as 30 seconds, 1 minute, 2 minutes, 5 minutes, 10 minutes, 30 minutes, 1 or more and less than 24 hours, 1 day, 1 week, or 1 month, for example.

With the wheezing-related information display apparatus according to the embodiment, as information indicating the temporal change in the frequency of the wheezing, the display processing unit displays a frequency of processing unit periods in which it was determined by the determination processing unit that wheezing is included, in each addition unit period including a plurality of the processing unit periods. Accordingly, the user can be made aware of the temporal change in the frequency of the wheezing in each of the addition unit periods.

The wheezing-related information display apparatus according to an embodiment further includes:

an addition processing unit configured to add up lengths of processing unit periods in which it was determined that the wheezing was included, in the addition unit periods, and obtain the result as a wheezing period,

wherein the display processing unit displays the information indicating the temporal change in the frequency of the wheezing as a bar graph indicating percentages occupied by the wheezing periods in bars with a certain length that correspond to the addition unit periods.

With the wheezing-related information display apparatus according to the embodiment, the addition processing unit adds up the lengths of the processing unit periods, in the addition unit periods, in which it was determined that wheezing was included, and obtains the result as the wheezing period. The display processing unit displays the information indicating the temporal change as a bar graph indicating percentages occupied by the wheezing periods in bars with a certain length that correspond to the addition unit periods. Accordingly, by looking at the bar graph, the user can, through vision, intuitively find out the frequency of the wheezing in the addition unit periods.

With the wheezing-related information display apparatus according to an embodiment, the display processing unit displays a plurality of bars corresponding to the addition unit periods in parallel alignment on the display screen.

In the wheezing-related information display apparatus according to the embodiment, the display processing unit displays multiple bars corresponding to the addition unit periods in parallel alignment on the display screen. Accordingly, the user can, through vision, intuitively find out the temporal change in the frequency of the wheezing in each of the addition unit periods.

With the wheezing-related information display apparatus according to an embodiment, in each of the processing unit periods, the addition processing unit converts the breathing sound signal into a frequency space to acquire a frequency spectrum of the breathing sound, classifies the power of the wheezing sound into a plurality of levels based on the area of a dominant peak that has the largest area in a graph of frequency with respect to sound pressure among a plurality of peaks in the frequency spectrum, and adds up the lengths of the processing unit periods in which it was determined that the wheezing is included, in each of the classified stages, and based on the addition performed by the addition processing unit, the display processing unit displays the wheezing periods divided into the levels in the bars corresponding to the addition unit periods.

Here, the above-described “peaks” in the frequency spectrum refer to peaks of sound intensity (sound pressure). The “areas” of the peaks in the frequency spectrum indicate the “areas” of peaks on a graph of frequency with respect to sound pressure. Also, if background noise exists in the graph of frequency with respect to sound pressure, the “areas” indicate the substantial “areas” of the peaks from which the background noise has been removed (the same follows for later-described “heights” and “widths” of the peaks as well).

The area of a peak in the frequency spectrum corresponds to the energy of that peak (frequency component). Accordingly, it can be said that the area of a peak, and in particular, the area of the dominant peak, corresponds to the severity of the wheezing. Here, with the wheezing-related information display apparatus according to the embodiment, the addition processing unit acquires the frequency spectrum of the breathing sound by converting the breathing sound into the frequency space in each of the processing unit periods, classifies the power of the breathing sound into a plurality of levels based on the area of a peak in the frequency spectrum, and adds up the lengths of the processing unit periods in which it was determined that the wheezing is included, for each of the classified levels. The display processing unit displays the wheezing periods divided into the levels in the bars corresponding to the addition unit periods based on the addition performed by the addition processing unit. Accordingly, by looking at the bar graph, the user can, through vision, intuitively find out the temporal change in the frequency of the wheezing in each addition unit period, as well as the temporal change in the power of the wheezing, or in other words, the severity of the wheezing, in each addition unit period.

The wheezing-related information display apparatus according to an embodiment further includes a medication administration information input unit for inputting information relating to administration of medication to the measurement subject,

wherein the display processing unit displays the information related to the administration of medication for each addition unit period on the display screen, along with the bar graph.

Here, “information related to administration of medication” includes information indicating the type of medication administered and the medication administration time, for example.

With the wheezing-related information display apparatus of the embodiment, the user inputs information related to administration of medication to the measurement subject using the medication administration information input unit. The display processing unit displays the information related to the administration of medication in each of the addition unit periods on the display screen, along with the bar graph. Accordingly, the user can, through vision, intuitively find out the frequency of the wheezing in the addition unit periods, as well as the information indicating the administration of medication. Accordingly, the user or a doctor who has been shown the display screen can easily determine whether or not the administration of medication to the measurement subject had an effect (a reduction in the frequency of wheezing).

The wheezing-related information display apparatus according to an embodiment further includes:

a phase identification unit configured to identify the breathing cycle of the measurement subject with a distinction made between an expiratory phase and an inspiratory phase, based on the breathing sound signal acquired by the breathing sound detection unit;

a phase instruction input unit configured to input an instruction to select one or both of the expiratory phase and the inspiratory phase of the breathing sound signal; and

a sound recording unit configured to record the phase of the breathing sound signal instructed by the phase instruction input unit.

With the wheezing-related information display apparatus according to the embodiment, the phase identification unit identifies the breathing cycle of the measurement subject with a distinction made between the expiratory phase and the inspiratory phase, based on the breathing sound signal acquired by the breathing sound detection unit. In response to a request made by the doctor, for example, the user uses the phase instruction input unit to input an instruction to select one or both of the inspiratory phase and the expiratory phase in the breathing sound signal. Upon doing so, the sound recording unit records the phase of the breathing sound signal instructed by the phase instruction input unit. Accordingly, when the doctor listens to the recorded content of the wheezing during the next medical examination, the user can have the doctor listen to the recorded content of the phase of the breathing cycle requested by the doctor.

### Advantageous Effects of the Invention

As is evident from the description above, with the wheezing-related information display apparatus of the present invention, it is possible to display the temporal change in the frequency of the wheezing included in the breathing sound of the measurement subject.

### BRIEF DESCRIPTION OF DRAWINGS

FIG. 1 is a drawing showing a schematic block configuration of a wheezing detection system according to an embodiment of the invention.

FIG. 2(A) is a diagram showing the exterior of a wheezing detector included in the above-mentioned wheezing detection system. FIG. 2(B) is a diagram showing an enlarged view of the exterior of a main body of the wheezing detector.

FIG. 3 is a diagram showing a block configuration of the main body of the wheezing detector.

FIG. 4 is a diagram showing a block configuration of a smartphone included in the wheezing detection system.

FIG. 5(A) is a diagram showing a mode in which the wheezing detector is attached to an infant serving as a measurement subject. FIG. 5(B) is a diagram showing a mode in which the wheezing detector is operated via the smartphone.

FIG. 6 is a diagram showing both a breathing sound signal detected by a microphone of the wheezing detector and a breathing flow amount signal detected by a breathing flow amount sensor.

FIG. 7 is a diagram illustrating a frequency spectrum obtained by converting the breathing sound signal into a frequency space.

FIG. 8 is a diagram illustrating a frequency spectrum of a breathing sound acquired in a certain processing unit period.

FIG. 9 is a diagram showing both temporal change in an L/D value of a dominant peak included in the frequency spectrum of the breathing sound and temporal change in the area of the dominant peak for a certain asthma patient.

FIG. 10 is a histogram showing the data frequency of L/D values for a normal breathing sound actually observed with no wheezing and the data frequency of L/D values for a breathing sound actually observed with wheezing.

FIG. 11 is a diagram schematically showing a method for expressing temporal change in the frequency of wheezing.

FIG. 12 is a diagram showing an example in which a bar graph expressing temporal change in the frequency of wheezing is displayed on a display screen of a smartphone.

FIG. 13 is a diagram showing both a breathing sound signal detected by a microphone of the wheezing detector and an envelope curve calculated for the breathing sound signal.

FIG. 14 is a diagram showing both the envelope curve shown in FIG. 13 and the breathing flow amount signal shown in FIG. 6.

FIG. 15(A) is a diagram showing an initialization menu screen for a “wheezing checker” program installed on the smartphone. FIG. 15(B) is a diagram showing a screen displayed when a “medication administration time” switch is pressed in FIG. 15(A).

FIG. 16(A) is a diagram showing a screen displayed when a “medication A” switch and a “medication B” switch are pressed in FIG. 15(B). FIG. 16(B) is a diagram showing an example in which both a bar graph indicating temporal change in the frequency of wheezing and information relating to administration of medication are displayed on the display screen of the smartphone.

FIG. 17 is a flowchart showing a procedure of operations performed by a user in the case of displaying the display example shown in FIG. 12 on the display screen of the smartphone.

FIG. 18 is a flowchart showing a procedure of operations performed by a user in the case of displaying the display example shown in FIG. 16(B) on the display screen of the smartphone.

FIG. 19 is a flowchart showing a procedure of operations performed by a user in the case of recording and playing back the breathing sound using the smartphone.

### DESCRIPTION OF EMBODIMENTS

Hereinafter, embodiments of the present invention will be described in detail with reference to the drawings.

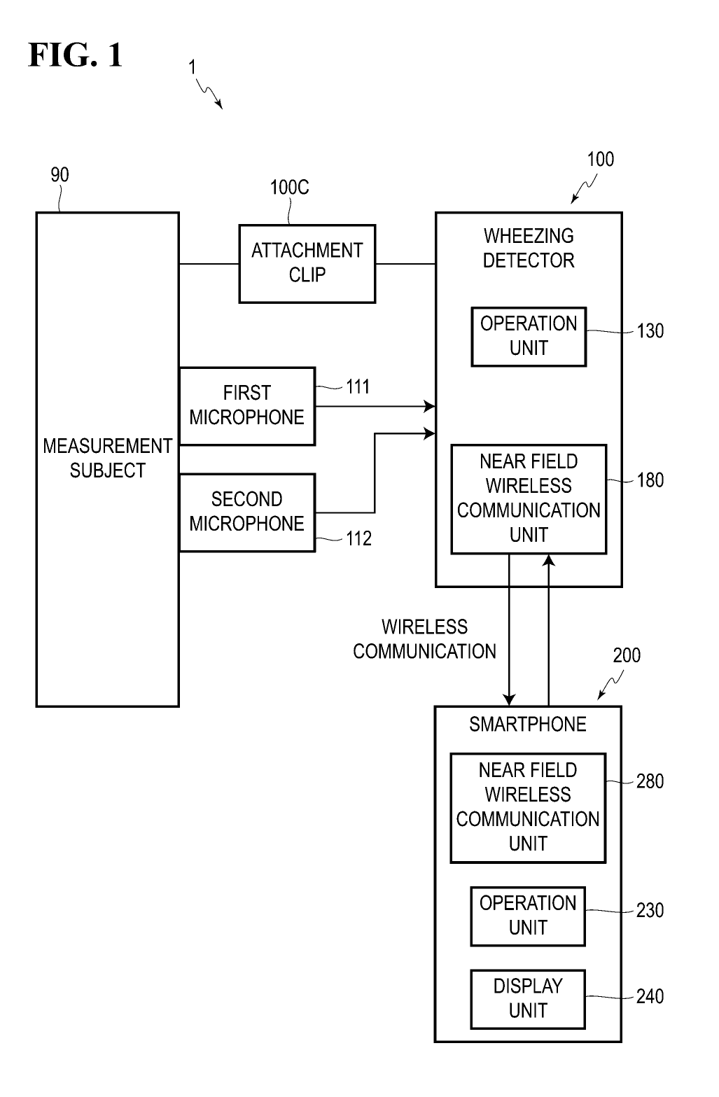

FIG. 1 shows a block configuration of a wheezing detection system (indicated overall by reference numeral 1), which is an embodiment of the wheezing-related information display apparatus of the invention. The wheezing detection system 1 includes a wheezing detector 100 and a smartphone 200. The wheezing detector 100 and the smartphone 200 can communicate with each other through wireless communication.

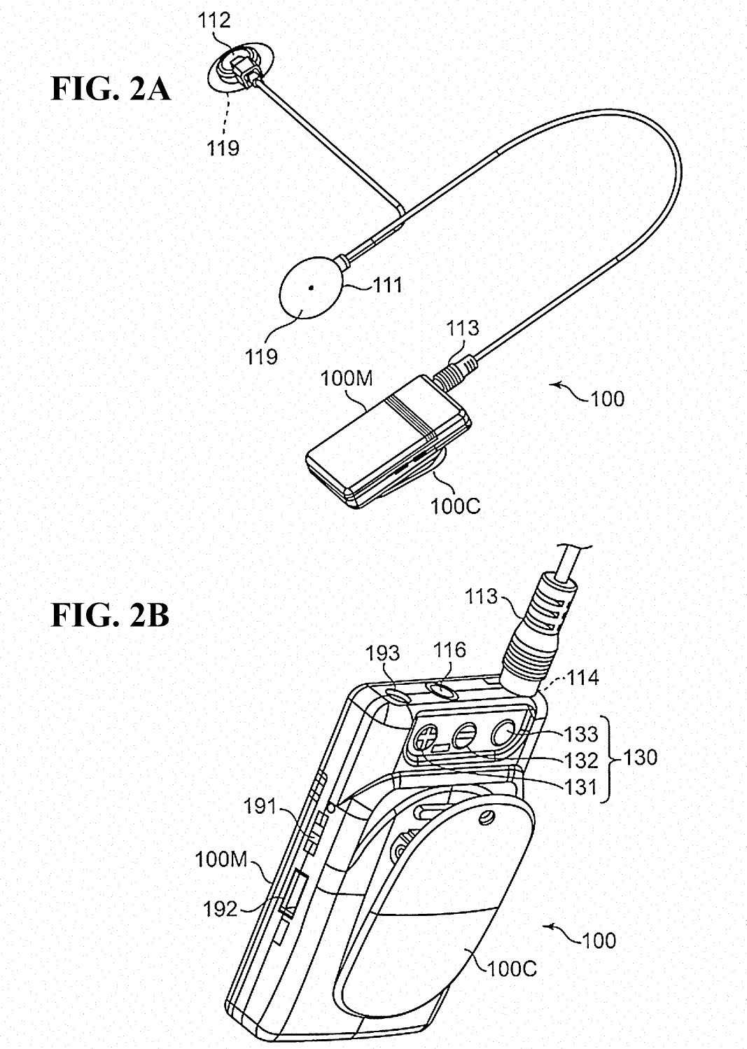

As shown in FIG. 2(A), the wheezing detector 100 includes a main body 100M, and a first microphone 111 and a second microphone 112 that are joined to the main body 100M via a microphone plug 113. In this example, the first microphone 111 and the second microphone 112 are in the form of stethoscopes that both have circular plate shapes (adhesive sheets 119 being provided on the recessed surfaces thereof). The first microphone 111 is to be adhered to the skin of the chest of the measurement subject, and the second microphone 112 is to be adhered to the clothing of the measurement subject.

As shown enlarged in FIG. 2(B), the main body 100M is provided with a clip 100C, a microphone terminal 114, an operation unit 130, a headphone terminal 116, a power source switch 191, a charging terminal 192, and a transmission state display LED (light emitting diode) 193.

The clip 100C is used to attach the main body 100M to the clothing of the measurement subject.

The microphone terminal 114 is used to receive output of the first microphone 111 and the second microphone 112 in a state in which the microphone plug 113 is inserted therein.

The operation unit 130 includes a volume increase button switch 131, a volume decrease button switch 132, and a communication switch 133. The volume increase button switch 131 is used to increase the volume of the sound output to headphones (not shown) via the headphone terminal 116. By contrast, the volume decrease button switch 132 is used to reduce the volume of the sound output to the headphones. The communication switch 133 is used to establish a connection for near field wireless communication between the main body 100M and the smartphone 200. In other words, when the communication switch 133 is pressed, a communication connection between the wheezing detector 100 and the smartphone 200 is established through a known protocol, and near field wireless communication becomes possible.

The power source switch 191 is used to switch on and off the power source of the wheezing detector 100.

The charging terminal 192 is used to charge a battery built into the main body 100M.

The communication state display LED 193 displays the state of communication between the wheezing detector 100 and the smartphone 200. Specifically, if near field wireless communication between the wheezing detector 100 and the smartphone 200 has not been connected, the LED 193 lights up with a red color. If the connection for near field wireless communication between the wheezing detector 100 and the smartphone 200 is in the process of being established, the LED 193 blinks with a green color. If the connection for near field wireless communication between the wheezing detector 100 and the smartphone 200 has been established, the LED 193 lights up with a green color. When the connection for near field wireless communication is established, the wheezing detector 100 enters a state of being able to operate (standby state) according to an instruction from the smartphone 200.

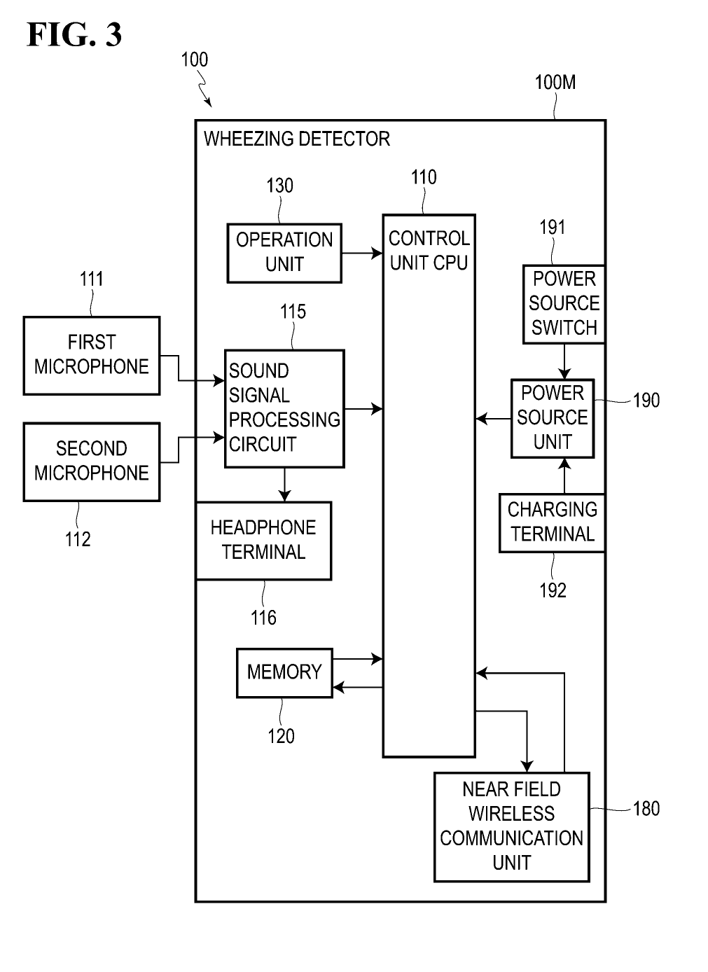

As shown in FIG. 3, the main body 100M of the wheezing detector 100 is provided with a control unit 110, a sound signal processing circuit 115, a memory 120, a near field wireless communication unit 180, and a power source unit 190 in addition to the above-mentioned headphone terminal 116, the operation unit 130, the power source switch 191, and the charging terminal 192.

The sound signal processing circuit 115 is composed of a CODEC-IC (CODEC integrated circuit), receives the output of the first microphone 111 and the output of the second microphone 112, subtracts the output of the second microphone 112 from the output of the first microphone 111, and outputs a breathing sound signal indicating the obtained difference to the control unit 110 and the headphone terminal 116. The user can confirm that the breathing signal has been obtained by connecting headphones (not shown) to the headphone terminal 116 and listening. Note that the first microphone 111, the second microphone 112, and the sound signal processing circuit 115 constitute a breathing sound detection unit.

The memory 120 includes a ROM (Read Only Memory) and a RAM (Random Access Memory). The ROM stores data of programs for controlling the wheezing detector 100. Also, the RAM stores setting data for setting various functions of the wheezing detector 100, data for a calculation result, and the like.

The control unit 110 includes a CPU (Central Processing Unit) and controls the units (including the memory 120 and the near field wireless communication unit 180) of the wheezing detector 100 in accordance with a program for controlling the wheezing detector 100, which is stored in the memory 120. In particular, the control unit 110 determines whether or not wheezing is included in the breathing sound of the measurement subject and creates image data indicating temporal change in the frequency of wheezing (this will be described in detail later).

In this example, the power source unit 190 includes a lithium ion battery (secondary battery) and supplies or stops supplying power to the units of the wheezing detector 100 according to switching on/off of the power source switch 191. The lithium ion battery can be charged via the charging terminal 192.

The near field wireless communication unit 180 performs wireless communication, and in this example, performs near field wireless communication (BT (Bluetooth (registered trademark)) communication and BLE (Bluetooth low energy) communication) with the smartphone 200 in accordance with control performed by the control unit 110. For example, information expressing a calculation result and the like is transmitted to the smartphone 200. Also, an operation instruction is received from the smartphone 200.

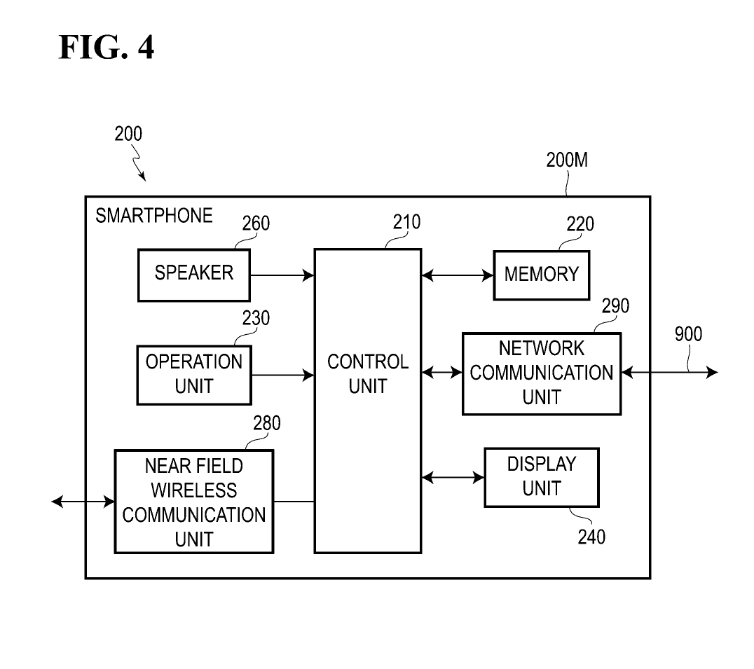

As shown in FIG. 4, the smartphone 200 includes a main body 200M, a control unit 210 mounted in the main body 200M, a memory 220, an operation unit 230, a display unit 240, a speaker 260, a near field wireless communication unit 280, and a network communication unit 290. The smartphone 200 has application software (referred to as a “wheezing checker” program) installed therein so as to cause a commercially-available smartphone to execute processing of information related to wheezing.

The control unit 210 includes a CPU and auxiliary circuits thereof, controls the units of the smartphone 200, and executes processing in accordance with a program and data stored in the memory 220. For example, based on an instruction input via the operation unit 230, the data input from the communication units 280 and 290 is processed, and the processed data is stored in the memory 220, displayed on the display unit 240, and is output via the communication units 280 and 290.

The memory 220 includes a RAM that is used as a work area needed for a program to be executed by the control unit 210, and a ROM for storing basic programs to be executed by the control unit 210. Also, a semiconductor memory (memory card, SSD (Solid State Drive)) or the like may be used as the storage medium of the auxiliary storage apparatus for assisting the storage region of the memory 220.

In this example, the operation unit 230 is composed of a touch panel provided on the display unit 240. Note that a keyboard or other hardware operation device may be included.

In this example, the display unit 240 includes a display screen composed of an LCD (liquid crystal display element) or an organic EL (electroluminescence) display. The display unit 240 displays various images on the display screen in accordance with control performed by the control unit 210.

The speaker 260 generates various sounds such as audio and an alarm sound serving as a warning, in accordance with control performed by the control unit 210.

The near field wireless communication unit 280 performs wireless communication, and in this example, near field wireless communication (BT communication and BLE communication) with the wheezing detector 100 in accordance with control performed by the control unit 210. For example, an operation instruction is transmitted to the wheezing detector 100. Also, information or the like indicating the calculation result is received from the wheezing detector 100.

The network communication unit 290 can transmit the information from the control unit 210 to another apparatus via the network 900, receive information transmitted via the network 900 from another apparatus, and transfer the received information to the control unit 210.

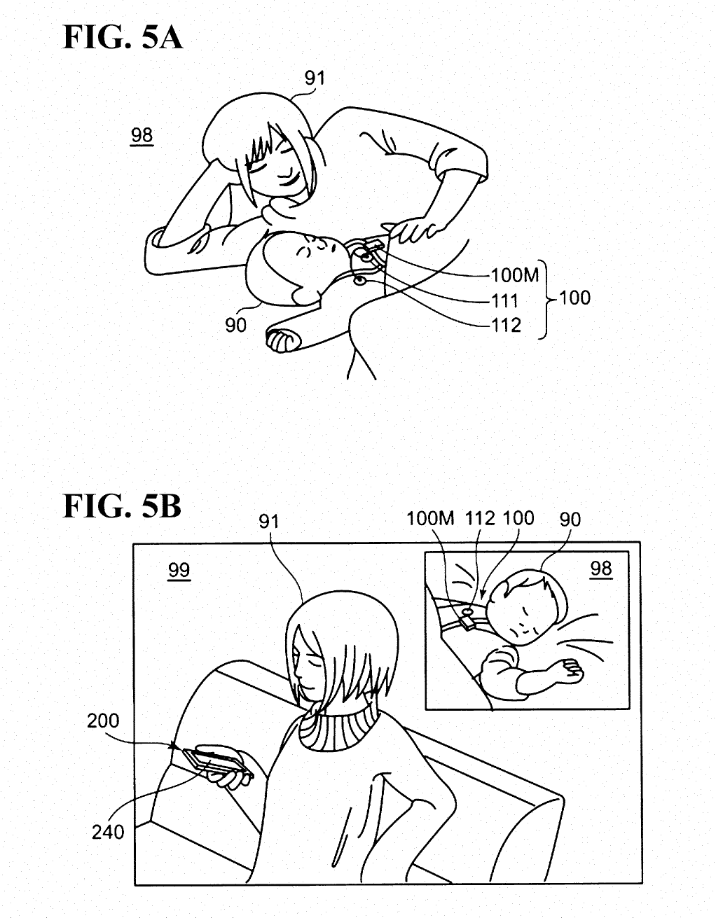

FIG. 5(A) illustrates a mode in which the wheezing detector 100 is attached to an infant 90 serving as the measurement subject. In this example, the infant 90 is laying down in a child's room 98, and the main body 100M of the wheezing detector 100 is attached to a sleeve of the clothing (in this example, pajamas) of the infant 90 via the clip 100C (see FIG. 2). The first microphone 111 is attached to the skin of the chest of the infant 90 with an adhesive sheet 119 provided on a circular plate. Also, the second microphone 112 is attached to the clothing (in this example, pajamas) of the infant 90. Note that the second microphone 112 may be attached to the skin of a part (a part having little influence on the breathing sound, such as a shoulder) located away from the chest and the respiratory organ of the infant 90.

First Operation Example

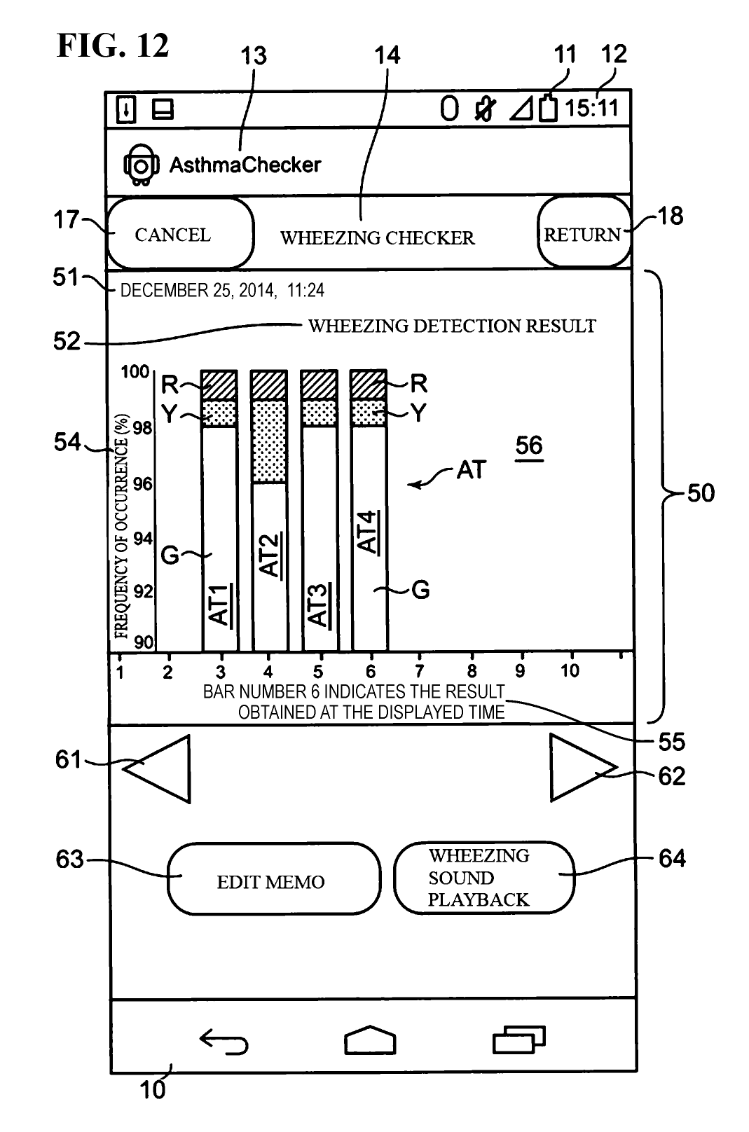

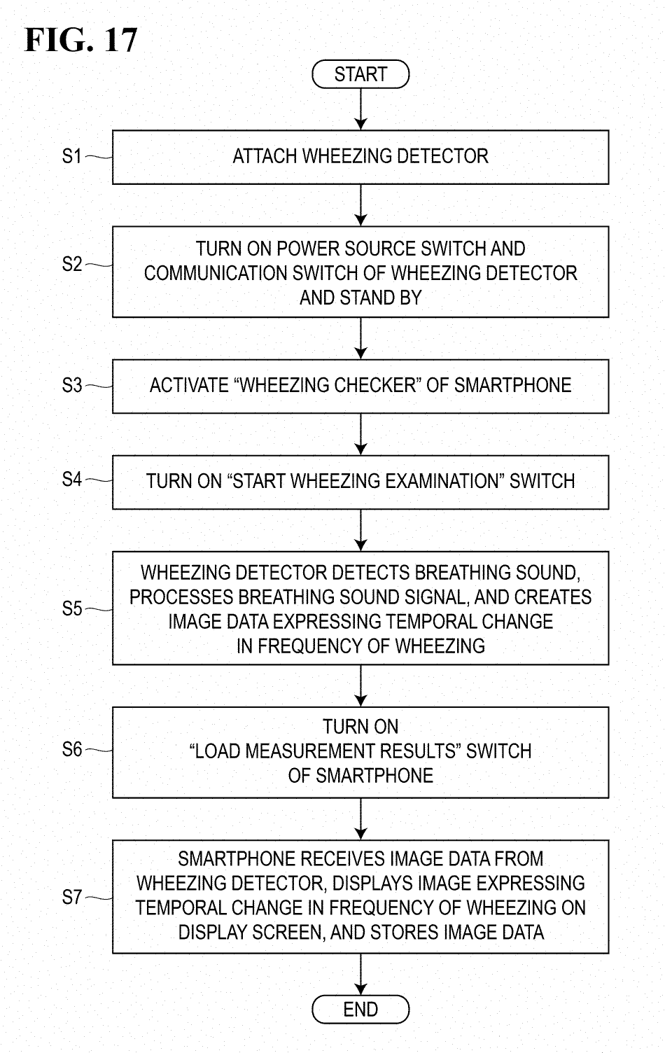

FIG. 17 shows an operation procedure by which the user (in this example, the mother of the infant 90) 91 uses the wheezing detection system 1 to cause the display screen of the smartphone 200 to display temporal change in the frequency of wheezing of the infant 90 (in this example, bar graph AT shown in FIG. 12).

(1) The user 91 attaches the wheezing detector 100 to the infant 90 as shown in FIG. 5(A) (step S1 in FIG. 17), presses the power source switch 191 and the communication switch 133 of the wheezing detector 100, and puts the wheezing detector 100 in the standby state (step S2 in FIG. 17).

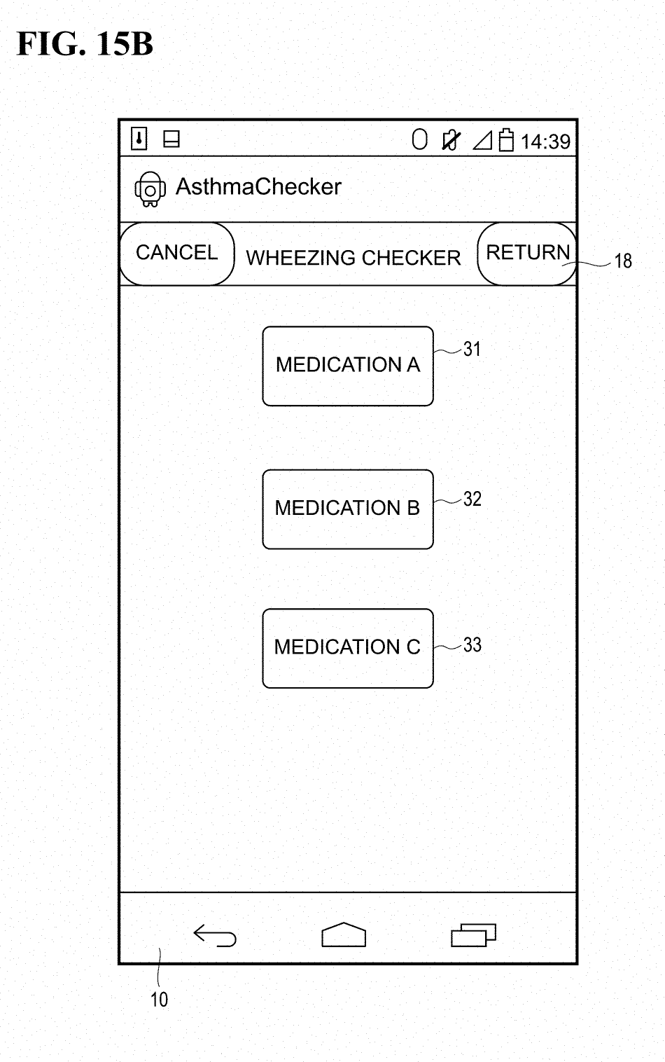

(2) Next, in a living room 99 that is different from the child's room 98, for example, as shown in FIG. 5(B), the user 91 activates a “wheezing checker” program installed in the smartphone 200 (step S3 in FIG. 17). Then, the user 91 presses a “start wheezing examination” switch (indicated by reference numeral 23 in later-described FIG. 15(A)) displayed on the display screen of the smartphone 200, thereby instructing the wheezing detector 100 to start measurement (step S4 in FIG. 17).

(3) Upon doing so, the wheezing detector 100 detects the breathing sound of the infant 90, the breathing sound signal is processed, and image data expressing the temporal change in the frequency of wheezing is created (step S5 in FIG. 17).

i) Specifically, first, the first microphone 111 detects mainly the breathing sound passing through the bronchial tubes of the infant 90, and the second microphone 112 detects mainly environmental sounds in the surroundings of the infant 90. The sound signal processing circuit 115 receives the output of the first microphone 111 and the output of the second microphone 112, subtracts the output of the second microphone 112 from the output of the first microphone 111, and outputs a breathing sound signal (indicated by reference numeral BS) in a time series indicating the obtained difference to the control unit 110. Accordingly, noise components in the surroundings of the infant 90 are removed from the breathing sound signal BS.

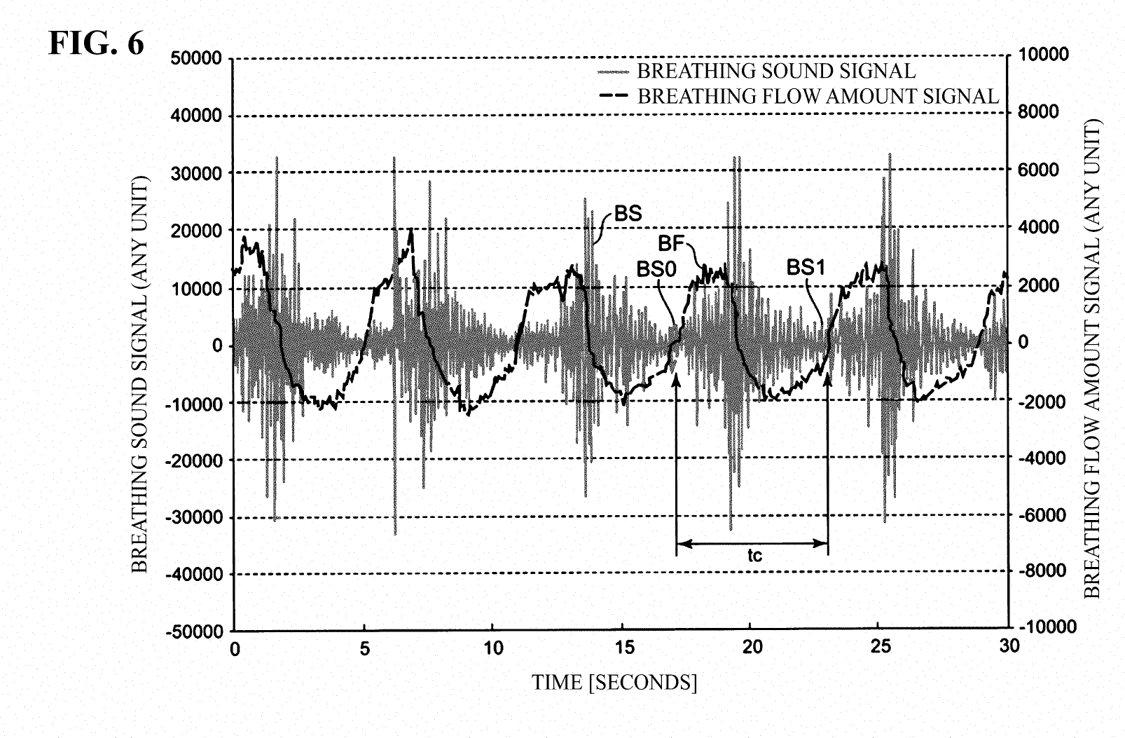

FIG. 6 illustrates the breathing sound signal BS obtained using the sound signal processing circuit 115 of the wheezing detector 100. Note that in FIG. 6, a breathing flow amount signal BF output by a breathing flow amount sensor (not included in the wheezing detection system 1) is also shown for reference. The positive side of the breathing flow amount signal BF indicates the flow amount for expiration and the negative side indicates the flow amount for inspiration.

ii) Next, the control unit 110 functions as a determination processing unit and determines whether or not wheezing is included in the breathing sound based on the breathing sound signal in each predetermined processing unit period (indicated by reference numeral tu; in this example, tu=0.05 seconds).

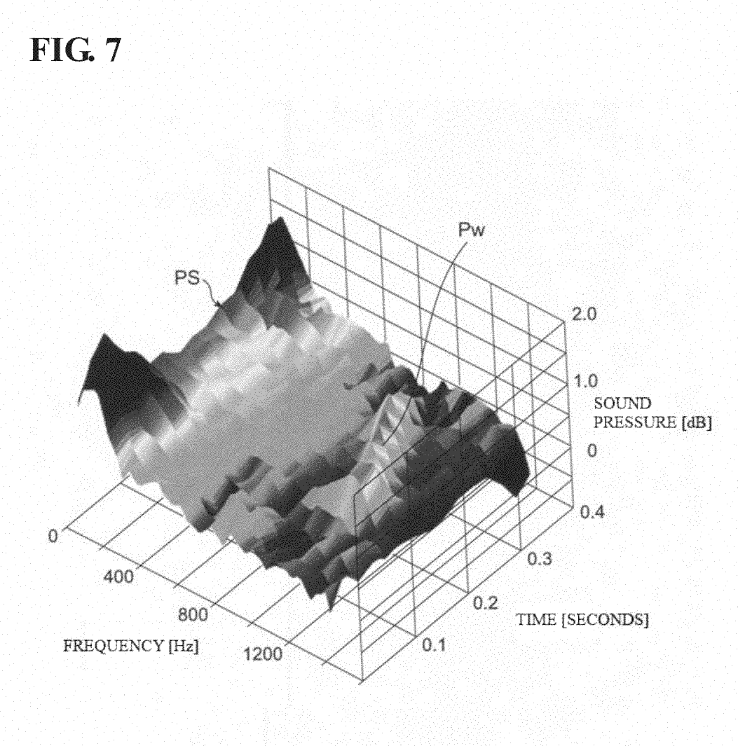

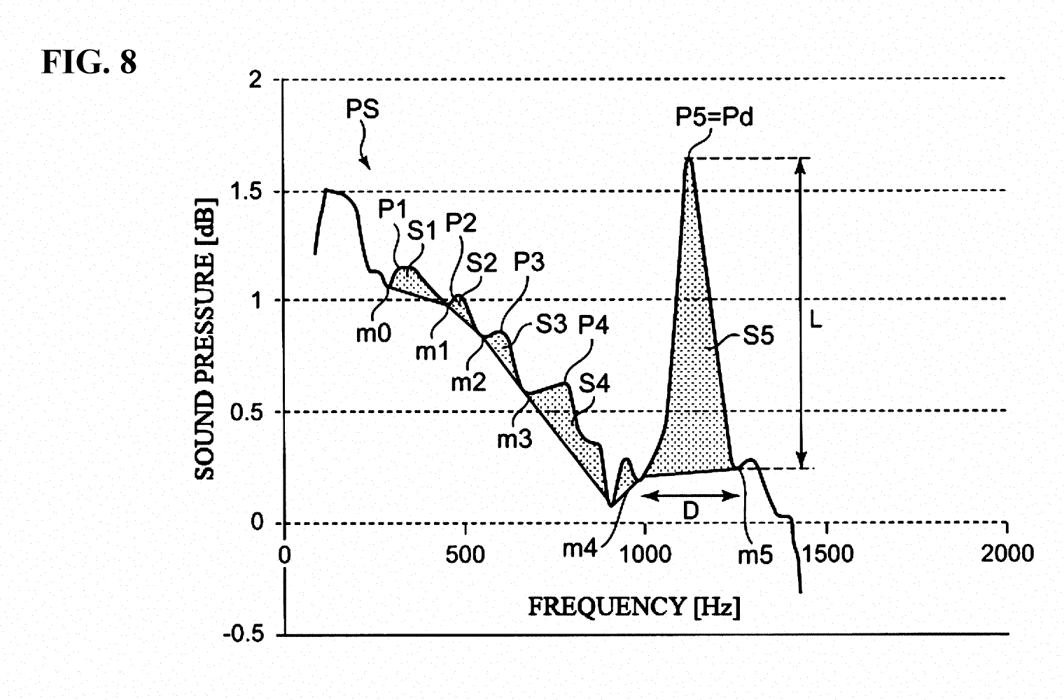

Here, FIG. 7 illustrates a frequency spectrum PS obtained by the control unit 110 converting the breathing sound signal BS into a frequency space in each processing unit period tu. Also, FIG. 8 illustrates a frequency spectrum PS for a breathing sound acquired in a certain processing unit period tu (in this example, corresponds to a processing unit period with a time of 0.05 seconds in FIG. 7). In the analysis performed by the inventor, the whistle-like sound Pw of wheezing (see FIG. 7) is characterized in that, as shown in FIG. 8, the width D of the peak of its frequency spectrum PS is relatively narrow (close to being monotone). Also, the wheezing sound is characterized in that it includes several peaks with relatively narrow widths D (e.g., see FIG. 6 in US 2011/0125044 A1). Accordingly, in order to accurately detect wheezing, the widths D of the peaks in the frequency spectrum PS should be used in the determination in some way. In view of this, with the wheezing detector 100, the control unit 110 determines whether or not peaks in the frequency spectrum PS indicates wheezing based on the heights L and the widths D of the peaks. More specifically, the control unit 110 obtains the ratios between the heights L and the widths D of the peaks (L/D; indicates the steepnesses of the peaks), and determines whether or not wheezing is indicated based on whether or not the ratios (L/D) are greater than a pre-determined first threshold (indicated by reference sign a; in this example, α=0.35).

Note that if background noise exists in the graph of frequency with respect to sound pressure, the heights L and the widths D of the peaks indicate the substantial heights L and the widths D of the peaks from which the background noise has been removed. For example, in the example shown in FIG. 8, it is thought that the heights L and the widths D are the substantial heights L and widths D of peaks P1, P2, P3, P4, and P5, which are portions that exceed line segments connecting local minimums m0, m1, m2, m3, m4, m5, . . . obtained in the graph of frequency with respect to sound pressure. Also, for the later-described areas of the peaks, substantial areas S1, S2, S3, S4, S5, . . . of the portions exceeding the line segments connecting the local minimums are indicated.

In the analysis performed by the inventor, the whistle-like sound Pw (see FIG. 7) of wheezing often observed in the case of infantile asthma is a sound having peaks with relatively narrow widths D (close to being monotone), with a frequency in a range of approximately 900 Hz to 1200 Hz. In view of this, with the asthma detector 100, the control unit 110 determines whether or not wheezing is indicated only for peaks having frequencies within the range of 200 Hz to 1500 Hz in the frequency spectrum PS. Accordingly, it is possible to detect whether or not wheezing including whistle-like wheezing that is often observed in the case of infantile asthma is included in the breathing sound of a measurement subject, in addition to the wheezing sound. On the other hand, sounds outside of the 200 Hz to 1500 Hz range are not thought of as being wheezing, and therefore are not handled in the determination.

Furthermore, the dominant peak Pd (in this example, peak P5) that has the largest area in the graph of frequency with respect to sound pressure (FIG. 8) among the multiple peaks P1, P2, P3, . . . in the frequency spectrum corresponds to the peak with the largest energy. Accordingly, the dominant peak Pd determines whether or not wheezing is included in the processing unit period tu. In view of this, in the wheezing detector 100, the control unit 110 determines whether or not wheezing is indicated based on only the dominant peak Pd that has the largest area in the graph of frequency with respect to sound pressure (FIG. 8) among the multiple peaks P1, P2, P3, . . . in the frequency spectrum PS.

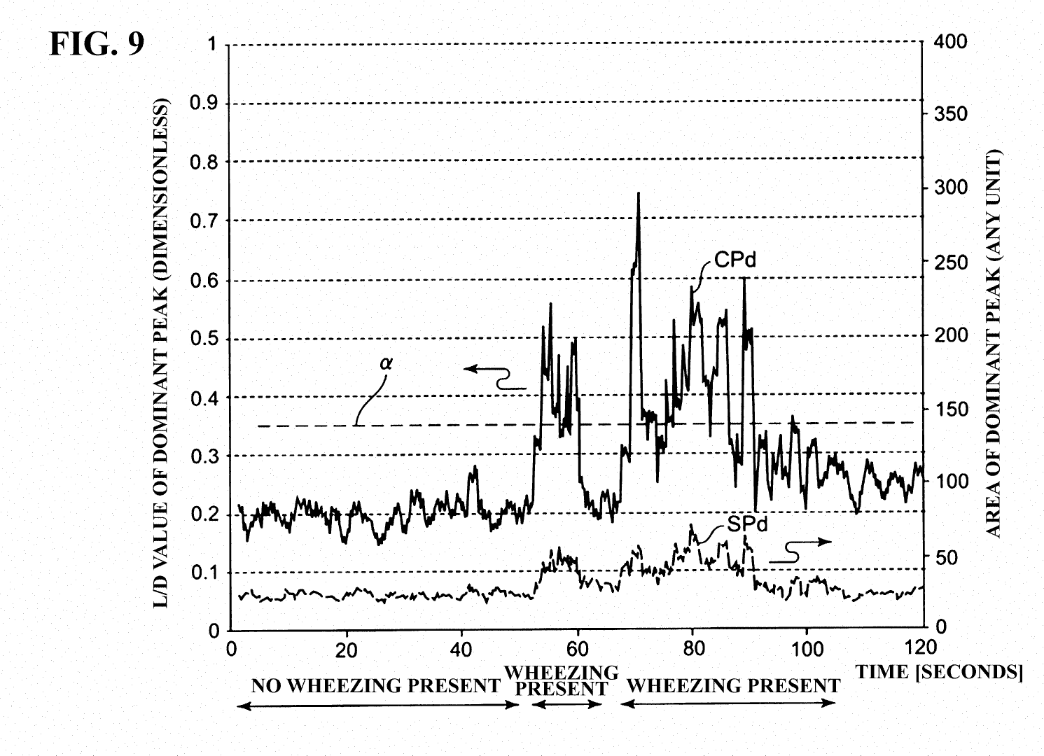

For example, FIG. 9 shows both temporal change CPd in the L/D value of the dominant peak Pd included in the frequency spectrum of the breathing sound of a certain asthma patient, and temporal change Spd in the area of the dominant peak Pd. Periods with no wheezing and periods with wheezing that are actually observed are specified on the time axis (horizontal axis). As can be understood from FIG. 9, it is understood that in the periods with no wheezing that were actually observed, the L/D value of the dominant peak Pd is less than or equal to the threshold value α, whereas in the periods with wheezing that were actually observed, the L/D value of the dominant peak Pd approximately exceeds the threshold value α. Also, accompanying this, in the periods with wheezing, the area of the dominant peak Pd is larger.

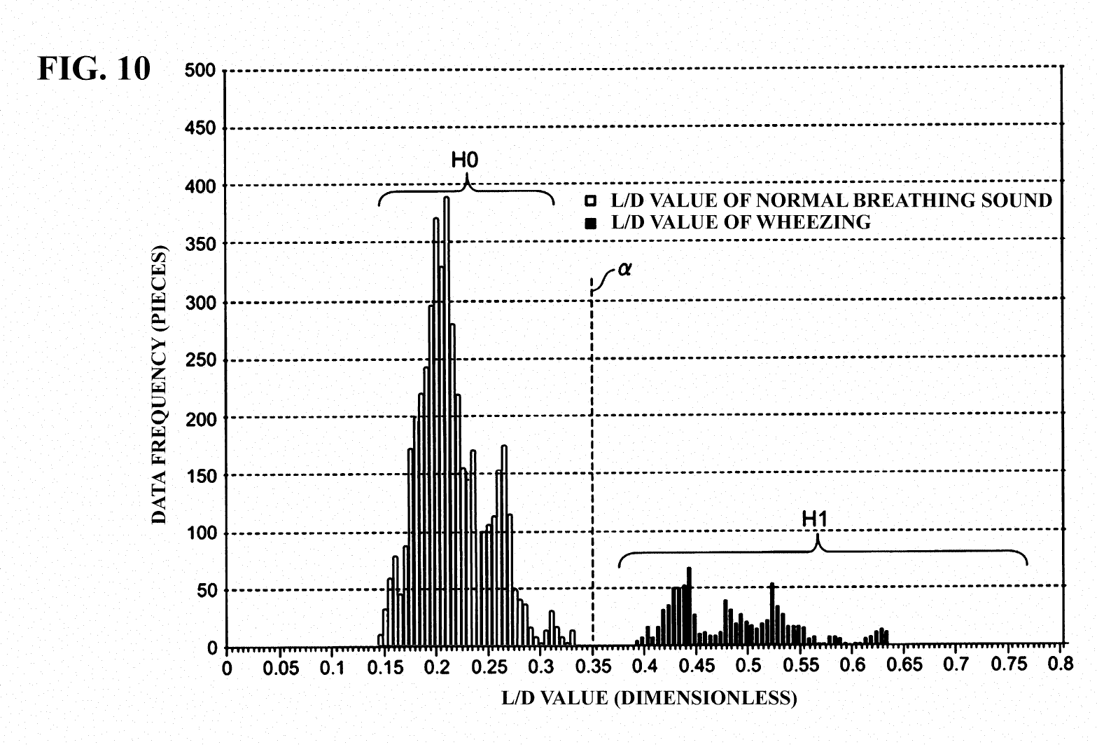

Also, FIG. 10 shows, as a histogram, the data frequency of the L/D values for a normal breathing sound actually observed as not including wheezing, and the data frequency of the L/D values for a breathing sound actually observed as including wheezing. As can be understood from FIG. 10, it is understood that data group H0 of L/D values for the normal breathing sound is less than or equal to the threshold value α, and data group H1 of L/D values for the breathing sound actually observed as including wheezing exceeds the threshold value α.

According to the results shown in FIGS. 9 and 10, it can be said that it is possible to accurately determine whether or not wheezing is included in the breathing sound of the measurement subject with the wheezing detector 100.

The determination results for each processing unit period tu, or in other words, the results of determining whether or not wheezing is included in the breathing sound of the measurement subject are sequentially stored as binary data in the memory 120 and accumulated. For example, if wheezing is included in the breathing sound, 1 is stored, and if breathing is not included in the breathing sound, 0 is stored.

iii) Next, based on the above-described determination results, the control unit 110 functions as an addition processing unit, sets an addition unit period (e.g., 30 seconds) including multiple processing unit periods tu, and sequentially adds up the lengths of the processing unit periods tu, in each addition unit period, in which it was determined that wheezing was included.

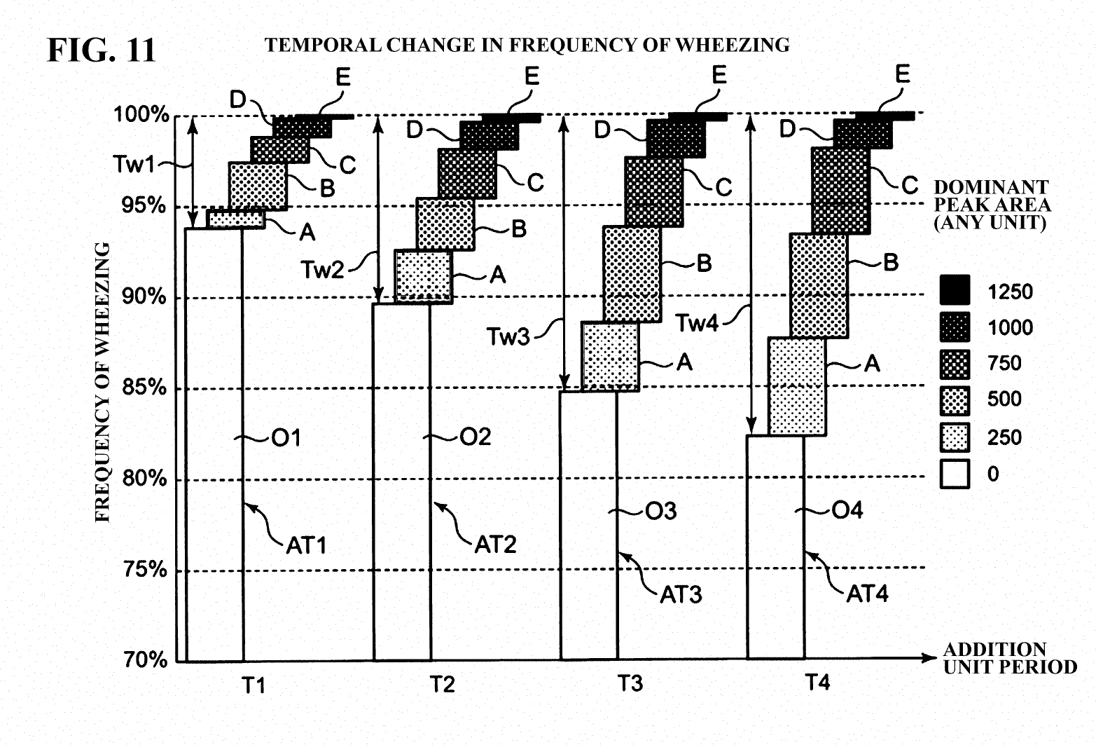

Specifically, as shown schematically in FIG. 11, in each addition unit period T1, T2, T3, T4, . . . , the lengths of the processing unit periods tu, in each processing unit period, in which it was determined that wheezing was included, are added up, and the results are obtained as wheezing periods Tw1, Tw2, Tw3, Tw4, . . . . Furthermore, information indicating temporal change in the frequency of wheezing is created as a graph indicating percentages that the wheezing periods Tw1, Tw2, Tw3, Tw4, . . . occupy in bars AT1, AT2, AT3, AT4, having a certain length that corresponds to the addition unit period. Note that the percentages of the periods (normal breathing periods) in which wheezing is not present in the addition unit periods are indicated by O1, O2, O3, O4, . . . .

In particular, in this example, in each addition unit period T1, T2, T3, T4, . . . , the power of the wheezing sound is classified into five levels A, B, C, D, and E based on the area (S5 in the example shown in FIG. 8) of the dominant peak Pd in the frequency spectrum Ps of the breathing sound, and the lengths of the processing unit periods to in which it was included that wheezing is included are added up for each of the classified levels A, B, C, D, and E. Level A is set such that the area of the dominant peak Pd is 0 or more and less than 250, level B is set such that the area of the dominant peak Pd is 250 or more and less than 500, level C is set such that the area of the dominant peak Pd is 500 or more and less than 750, level D is set such that the area of the dominant peak Pd is 750 or more and less than 1000, and level E is set such that the area of the dominant peak Pd is 1000 or more and less than 1250. Note that in the example shown in FIG. 11, in order to facilitate understanding, the percentages of the five levels A, B, C, D, and E are shown shifted sequentially to the right, but they may be shown straight as well.

The levels for classifying the power of the wheezing sound (i.e., the severity of the wheezing) are not limited to the five levels A, B, C, D, and E. For example, it might be easier for an average user who is not a medical professional to intuitively understand that the power of the wheezing sound is classified into three levels.

iv) In view of this, in the case of creating image data that is to be transmitted to the smartphone 200 in actuality, the control unit 110 functions as a display processing unit and sets the percentages obtained by combining the normal breathing periods and the periods in which the area of the dominant peak Pd is in a range of 0 or more and less than 250 to green G The percentages of the periods in which the area of the dominant peak Pd is in a range of 250 or more and less than 750 are made yellow Y. Also, the percentages of the periods in which the area of the dominant peak Pd is in the range of 750 or more are made red R. In response to this, the bars with certain lengths corresponding to the addition unit periods are displayed divided into three colors, namely green yellow Y, and red R. Also, the control unit 110 aligns the multiple bars AT1, AT2, . . . corresponding to the addition unit period in parallel so as to create image data indicating a bar graph (in this example, the bar graph AT shown in FIG. 12) indicating temporal change in the frequency of wheezing.

Note that in the example shown in FIG. 11, the addition unit period was set to be 30 seconds, but there is no limitation to this. The addition unit period can be set in various ways, such as 1 minute, 2 minutes, 5 minutes, 10 minutes, 30 minutes, one hour or more and less than 24 hours, one day, or one month. In the following example, the addition unit period is one hour.

Also, when the percentage of time for which the power of the wheezing sound has reached red R exceeds a pre-determined second threshold (indicated by reference sign (3; in this example, 0=5[%]) in an addition unit period, the control unit 110 functions as a warning generation unit that transmits an alarm signal as a warning to the smartphone 200 via the near field wireless communication unit 180.

(4) Next, the user 91 presses a “load measurement results” switch (indicated by reference sign 28 in later-described FIG. 15(A)) displayed on the display screen of the smartphone 200 and receives the image data from the wheezing detector 100 via the near field wireless communication unit 280 (in particular, BLE communication) (step S6 in FIG. 17). The received data is automatically stored and recorded in the memory 220 serving as the storage unit.

Upon doing so, the bar graph AT indicating the temporal change in the frequency of the wheezing illustrated in FIG. 12 is displayed on the display screen 10 of the smartphone 200.

Here, a battery remaining amount 11 and a current time 12 are displayed at the uppermost level of the display screen 10. Also, below that, an “AsthmaChecker” display 13 is provided as the name of the application software, and a “wheezing checker” display 14 is provided. A “cancel” switch 17 for inputting an instruction to end the “wheezing checker” program and a “return” switch 18 for inputting an instruction to return to the screen displayed immediately before the content of the display screen are provided on the left and right of the “wheezing checker” display 14. Furthermore, below that, a wheezing detection result display field 50 for displaying image data received from the wheezing detector 100 is provided.

A measurement time display (in this example, “December 25, 2014, 11:24”) 51 that displays the final measurement date and time, a field name display 52 that reads “wheezing detection results”, and an image data display region 56 are provided in the wheezing detection result display field 50. An order (in this example, “1, 2, 3, . . . ”) 55 of the addition unit periods is displayed on the horizontal axis in the image data display region 56. Also, directly below the horizontal axis, it is indicated that the data on the measurement date and time indicated in the measurement time display 51 is included in the sixth piece of data (bar AT 4) as “number 6 indicates the result obtained at the displayed time”. Furthermore, in the image data display region 56, as the vertical axis, the “frequency of occurrence (%)” is displayed in increments of 2%, e.g., 90%, 92%, 94%, . . . , 100%. Also, the bar graph AT indicating the temporal change in the frequency of the above-described wheezing is displayed inside of the image data display region 56.

The bar graph AT includes multiple bars AT1, AT2, . . . with a certain height, which each correspond to the addition unit period (in this example, one hour), in the order of the temporal change. The bars are indicated with three colors, namely green yellow Y, and red R. As described above, green G indicates a percentage obtained by combining a normal breathing period and a period in which the area of the dominant peak Pd is 0 or more and less than 250, or in other words, a percentage of time for which there is no or approximately no wheezing, in the addition unit period corresponding to the bar. Yellow Y indicates a percentage of time for which the area of the dominant peak Pd is 250 or more and less than 750, or in other words, a percentage of time for which wheezing is relatively small. Red R indicates a percentage of time for which the area of the dominant peak Pd is 750 or more, or in other words, a percentage of time for which wheezing is relatively large.

By looking at the bar graph AT, the user 91 can intuitively, through vision, find out the temporal change in each addition unit period of the severity of wheezing, along with the temporal change in each addition unit period of the frequency of wheezing.

For example, in the example of the bar graph AT shown in FIG. 12, in addition unit period number “3”, the percentage of time obtained by combining yellow Y and red R is about 2%, and therefore it can be understood that there was wheezing for about 1.2 minutes in one hour. Also, since the percentage of time for yellow Y and the percentage of time for red R are each about 1%, it can be understood that there was relatively small wheezing and relatively large wheezing for about the same percentages of time. Also, in addition unit period number “4”, the percentage of time obtained by combining yellow Y and red R is about 4%, and therefore it can be understood that there was wheezing for about 2.4 minutes in one hour. Also, the percentage of time for yellow Y is about 3% and the percentage of time for red R is about 1%, and therefore it can be understood that the percentage of time for relatively small wheezing increased to about three times the percentage of time for relatively large wheezing. Also, in addition unit periods number “5” and “6”, it can be understood that there has been a return to the approximate state of addition unit period number “3”.

Thus, by looking at the bar graph AT, the user 91 can, through vision, intuitively find out the temporal change in each addition unit period of the frequency of wheezing and the severity of wheezing included in the breathing sound of the infant 90 serving as the measurement subject. Also, the information expressing the bar graph AT is automatically stored in the memory 220. Accordingly, by loading the information from the memory 220 and causing the bar graph AT to be displayed on the display screen 10 the next time the infant 90 has a medical examination, the user 91 can show the doctor the temporal change in the frequency of wheezing and the severity of wheezing included in the breathing sound of the infant 90. As a result, the doctor can more easily diagnose whether or not the infant 90 has asthma and the severity of the asthma, and can easily create a treatment plan.

Also, the user 91 can understand whether the asthma is getting worse or better by viewing the state of the temporal change for each addition unit period of the frequency of wheezing and the severity of wheezing in the bar graph AT. For example, if the asthma is getting worse, an advance measure such as administering medication is possible, which leads to preventing the asthma from worsening.

On the display screen 10 of the smartphone 200, a direction key 61 according to which a target period displayed in the wheezing detection result display field 50 is moved up, and a direction key 62 according to which a target period is moved down are provided below the wheezing detection result display field 50. The user can select a target period displayed on the display screen 10 as the wheezing detection result by pressing the direction keys 61 and 62. Also, if the user presses the “edit memo” switch 63, a memo screen (not shown) is opened, and the user can manually input and record what he or she felt upon seeing the asthma detection results. Also, a “wheezing sound playback” switch 64 will be described later.

When the percentage of time for which the power of the wheezing sound reaches red R in the addition unit period exceeds the predetermined threshold β (=5 [%]), the smartphone 200 receives an alarm signal from the wheezing detector 100 via the near field wireless communication unit 280. Upon receiving the alarm signal, the control unit 210 of the smartphone 200 uses the speaker 260 to generate an alarm sound serving as a warning. With this alarm sound, the user 91 can be made aware of the fact that the symptoms of the infant 90 serving as the measurement subject have worsened, even if the user 91 is in a living room 99 separate from the child's room (where the infant 90 is lying) 98. Accordingly, it is possible to take a countermeasure such as administering medication to the infant 90. The warning is particularly advantageous in the case where the measurement subject is an infant 90, a critically ill patient, or the like, who has difficulty expressing intention.

Note that the warning is not limited to generation of an alarm sound using the speaker 260, and it is also possible to perform alarm display (not shown) on the display screen 10, or vibrate using a vibrator (not shown) that performs notification of signal reception.

Second Operation Example

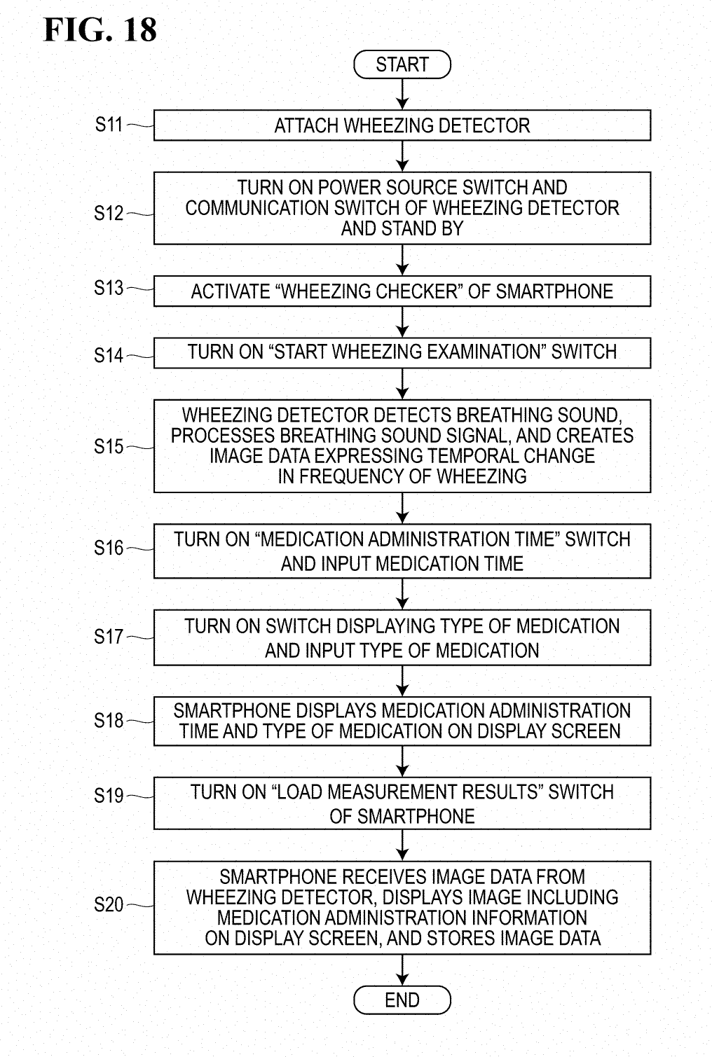

FIG. 18 shows an operation procedure for the user 91 to display, on the display screen of the smartphone 200, the bar graph AT indicating the temporal change in the frequency of wheezing of the infant 90 and the information relating to administration of medication, according to the wheezing detection system 1.

(1) Steps S11 to S15 in FIG. 18 are carried out similarly to steps S1 to S5 in FIG. 17.

Here, FIG. 15(A) illustrates an initialization menu screen displayed on the display screen 10 when the user 91 activates the “wheezing checker” program installed in the smartphone 200 in step S13 of FIG. 18.

On the initialization menu screen shown in FIG. 15(A), a state 16 of “not connected” or “connected” of BT communication and BLE communication with the wheezing detector 100 performed by the near field wireless communication unit 280 is displayed between the “Asthma Checker” display 13 and the “wheezing checker” display 14. Also, below the “wheezing checker” display 14, a “reserve” switch 21, a “setting” switch 22, a “start wheezing examination” switch 23, a “stop wheezing examination” switch 24, a “start recording” switch 25, a “stop recording” switch 26, a “setting screen” switch 27, a “load measurement results” switch 28, a “medication administration time” switch 30, and a phase selection switch 40 serving as a phase instruction input unit are provided.

The “reserve” switch 21 is used in order for the user 91 to reserve a period of time, such as from 12/26/2014 9:00 PM to 12/27/2014 7:00 AM, during which measurement is to be performed by the wheezing detector 100. The “setting” switch 22 is used to set the condition (in this example, the threshold value β) under which the wheezing detector 100 generates the above-described alarm signal, and to set whether or not to generate the alarm sound, whether or not to perform alarm display, whether or not to perform automatic recording, and the like when the smartphone 200 receives the alarm signal. The “start wheezing examination” switch 23 is used to instruct the wheezing detector 100 to start measurement. The “stop wheezing examination” switch 24 is used to instruct the wheezing detector 100 to stop measurement. The “start recording” switch 25 is used to instruct the wheezing detector 100 to transmit the breathing sound signal BS. The “stop recording” switch 26 is used to instruct the wheezing detector 100 to stop transmitting the breathing sound signal BS. The “setting screen” switch 27 is used to set an SSID (Service Set Identification) and an encryption key (password) between the near field wireless communication unit 280 of the smartphone 200 and the near field wireless communication unit 180 of the wheezing detector 100. The “medication administration time” switch 30 is used to input a time for administering medication (year, month, day, hour, minute). Note that the phase selection switch 40 will be described later.

(2) In this example, in the state in which the initialization menu screen shown in FIG. 15(A) is displayed on the display screen 10 of the smartphone 200, the user 91 presses the “medication administration time” switch 30 (step S16 in FIG. 18). Upon doing so, the time at which the “medication administration time” switch 30 was pressed is stored as the medication administration time (year, month, day, hour, minute) in the memory 220. Note that it is also possible to use a configuration in which when the user 91 presses the “medication administration time” switch 30, a screen for inputting the medication administration time opens, the user 91 inputs the medication administration time (year, month, day, hour, minute) in that screen, and when the user 91 presses the “medication administration time” switch 30 again, the input medication administration time is stored. When the medication administration time is stored, the medication administration information input screen for inputting the type of medication that was administered, as illustrated in FIG. 15(B), is displayed on the display screen 10. In this example, three types of switches, namely a “medication A” switch 31, a “medication B” switch 32, and a “medication C” switch 33 are displayed. Note that in actuality, specific medication names designated in a prescription by a doctor are registered in advance as the “medication A”, “medication B”, and “medication C” using a medication name registration screen (not shown). The specific medication names that were registered are displayed in the display locations for “medication A”, “medication B”, and “medication C” in FIG. 15(B). The switches 30, 31, 32, and 33 constitute medication administration information input units.

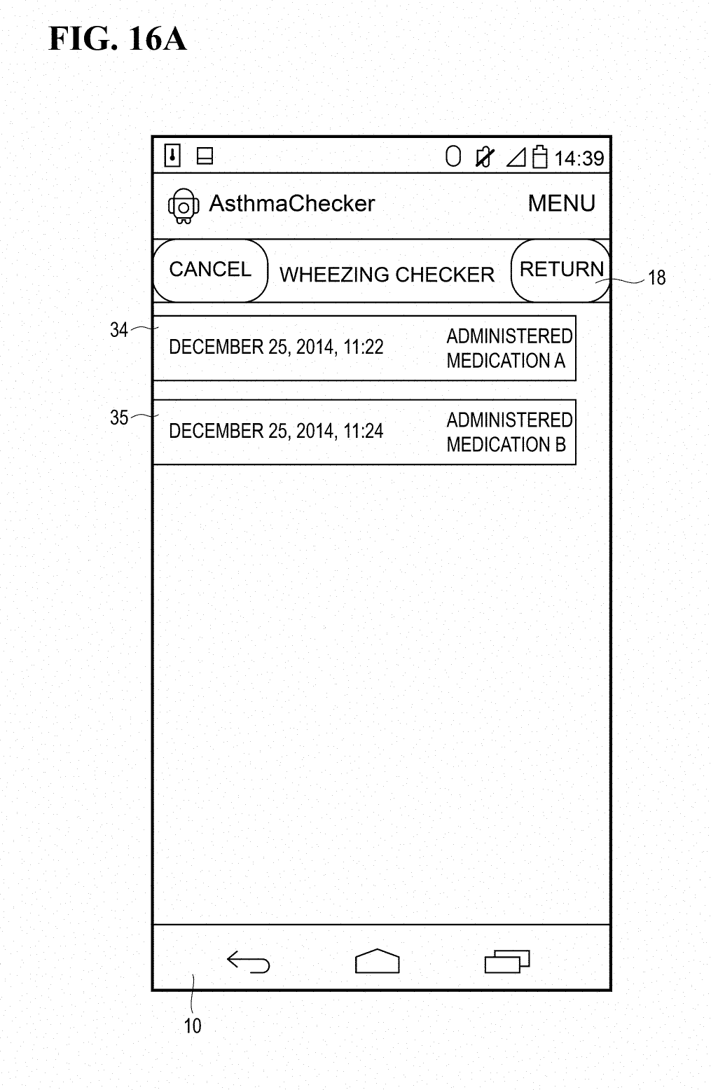

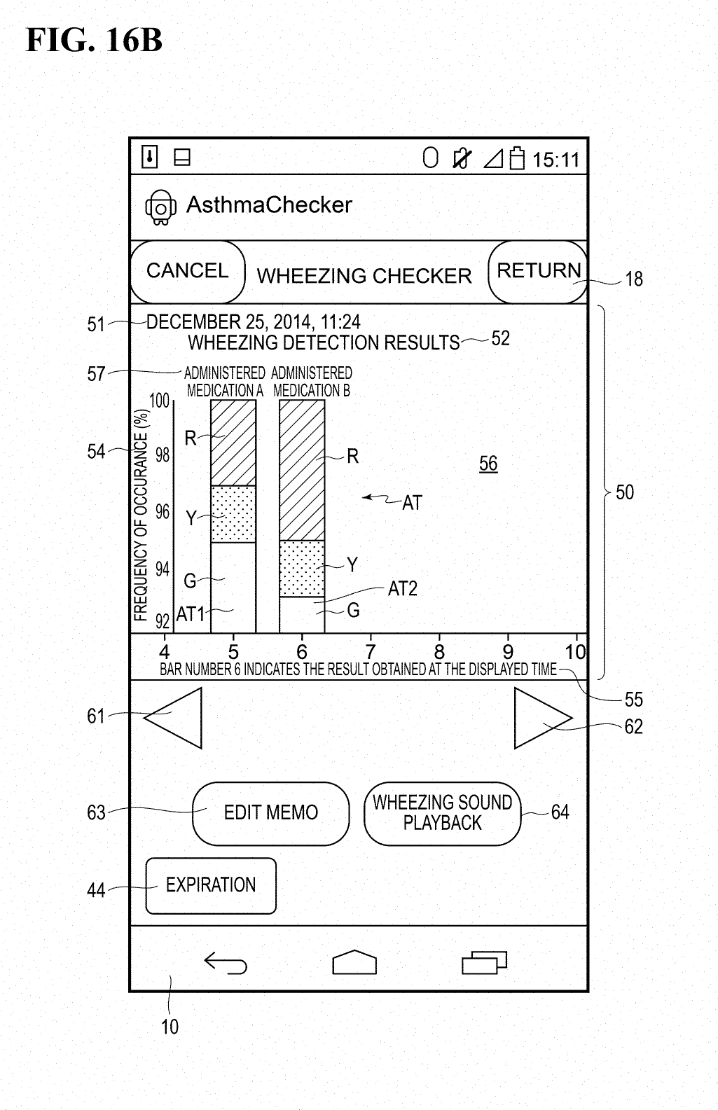

(3) In a state in which the medication administration information input screen shown in FIG. 15(B) is displayed, the user 91 pushes one of the medication switches and inputs the type of medication (step S17 in FIG. 18). For example, when the user 91 presses the “medication A” switch 31, a screen for checking that includes medication administration information 34, which says “December 25, 2014, 11:22, administered medication A” is displayed on the display screen 10 of the smartphone 200, as illustrated in FIG. 16(A) (step S18 in FIG. 18).

(4) Furthermore, when the user 91 presses the “return” switch 18 twice to return to the initialization menu screen shown in FIG. 15(A), presses the “medication administration time” switch 30, and presses the “medication B” switch 32 on the medication administration information input screen shown in FIG. 15(B), as illustrated in FIG. 16(A), a screen for checking that includes the medication administration information 35, which says “December 25, 2014, 11:24, medication B” is displayed along with the previous medication administration information 34. In other words, for each administration of medication, steps S16 to S18 in FIG. 18 are repeated.

(5) Thereafter, the user 91 presses the “return” switch 18 twice to return to the initialization menu screen shown in FIG. 15(A), presses the “load measurement result” switch 28 to receive image data from the wheezing detector 100 via the near field wireless communication unit 280 (in particular, BLE communication) (step S19 in FIG. 18). The received data is automatically stored and recorded in the memory 220 serving as the storage unit.

Upon doing so, the control unit 210 of the smartphone 200 functions as the display processing unit, and as shown in FIG. 16(B), the medication administration information for each addition unit period is displayed in the display screen 10 along with the bar graph AT indicating the temporal change in the frequency of wheezing (step S20 in FIG. 18). In the example shown in FIG. 16(B), “administered medication A” is displayed above the bar graph AT1 for the addition unit period number 5, and “administered medication B” is displayed above the bar graph AT1 for addition unit period number 6. The user 91 can, through vision, intuitively find out the frequency of wheezing in the addition unit period, information relating to medication, and in this example, find out that the medication A was given to the infant 90 in addition unit period number 5 and that the medication B was given to the infant 90 in addition unit period number 6. Accordingly, the user 91 or the doctor who has been shown the screen shown in FIG. 16(B) can easily determine whether or not the medication had an effect (a decrease or increase in the frequency of wheezing) on the infant 90.

For example, in the example shown in FIG. 16(B), regardless of the fact that there was “administration of medication A” in the addition unit period number 5 (where the percentage of the red R time was about 3%), the percentage of the red R time increased to about 5% in addition unit period 6, and the symptoms of the infant 90 worsened. For this reason, there is a high likelihood that “administration of medication A” was not effective. It might be better to determine the effect of “administration of medication B” after viewing addition unit period number 7 and onward.

Third Operation Example

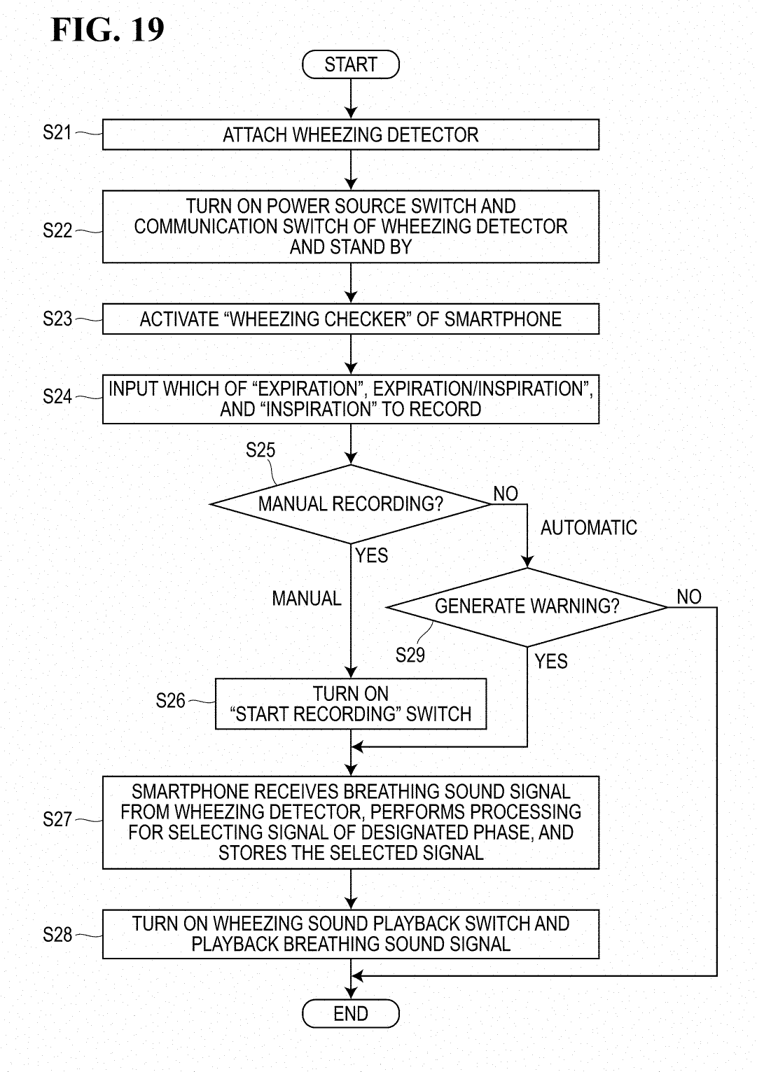

FIG. 19 shows a procedure of operations for the user 91 to use the wheezing detection system 1 to record the breathing sound of the infant 90 in the memory 220 of the smartphone 200.

(1) Steps S21 to S23 in FIG. 19 advance similarly to steps 51 to S3 in FIG. 17.

Here, in step S23 of FIG. 19, the initialization menu screen shown in FIG. 15(A) is displayed on the display screen 10 of the smartphone 200. A phase selection switch 40 serving as a phase instruction input unit is included on the initialization menu screen. When the breathing sound signal BS is to be recorded, the phase selection switch 40 includes an “expiration” switch 41 for selecting only the expiratory phase, an “expiration/inspiration” switch 42 for selecting both the expiratory phase and the inspiratory phase, and an “inspiration” switch 43 for selecting only the inspiratory phase.

(2) In a state in which the phase selection switch 40 is displayed on the display screen 10 of the smartphone 200, according to a request made by the doctor, for example, the user 91 presses one switch among the “expiration” switch 41, the “expiration/inspiration” switch 42, and the “inspiration” switch 43 according to which of only the expiratory phase, both the expiratory phase and the inspiratory phase, and only the inspiratory phase is to be recorded (step S24 in FIG. 19). Accordingly, the phase to be recorded is selected.

(3) Next, the user 91 determines whether to manually record or automatically record (step S25 in FIG. 19). For example, if the current wheezing symptoms of the infant 90 are severe and the user 91 wants to record the wheezing immediately, it is desirable to select manual recording. On the other hand, if the current wheezing symptoms of the infant 90 are favorable and the user 91 wants to record when the wheezing symptoms become severe, it is desirable to select automatic recording.

(4) In the case of performing manual recording (YES in step S25 of FIG. 19), the user 91 presses the “start recording” switch 25 on the initialization menu screen shown in FIG. 15(A) (step S26 of FIG. 19). Upon doing so, the control unit 210 of the smartphone 200 instructs the wheezing detector 100 to transmit the breathing sound signal BS via the near field wireless communication unit 280. On the other hand, in the case of performing automatic recording, the user 91 uses the “setting” switch 22 shown in FIG. 15(A) to set that “automatic recording” is to be performed. In the “automatic recording” mode, the control unit 210 of the smartphone 200 waits for the above-described alarm signal (indicates that the percentage of the time for which the addition unit period reaches red R has exceeded the threshold (3) from the wheezing detector 100 (step S29 in FIG. 19), and instructs the wheezing detector 100 to transmit the breathing sound signal BS via the near field communication unit 280 at the time of receiving the alarm signal (YES in step S29 of FIG. 19). In both the case of manual recording and the case of automatic recording, when the wheezing detector 100 receives the instruction to transmit the breathing sound signal BS from the smartphone 200, the wheezing detector 100 transmits the breathing sound signal BS to the smartphone 200 via the near field wireless communication unit 180 (in particular, BT communication).

(5) Upon receiving the breathing sound signal BS, in the smartphone 200, the control unit 210 functions as a sound recording unit and performs recording by storing the phase of the breathing sound signal BS selected with the phase selection switch 40 in the memory 220 (step S27 in FIG. 19).

More specifically, the control unit 210 of the smartphone 200 functions as the phase selection unit and detects the phases of the breathing sound signal BS as follows.

First, as shown in FIG. 6, the breathing sound signal BS has local minimums in synchronization with zero-crossing points at which the breathing flow amount signal BF transitions from negative (inspiration) to positive (expiration). Accordingly, the control unit 210 can obtain a breathing cycle tc of the measurement subject (in this example, the infant 90) by detecting the local minimums BS0, BS1, . . . of the breathing sound signal BS.

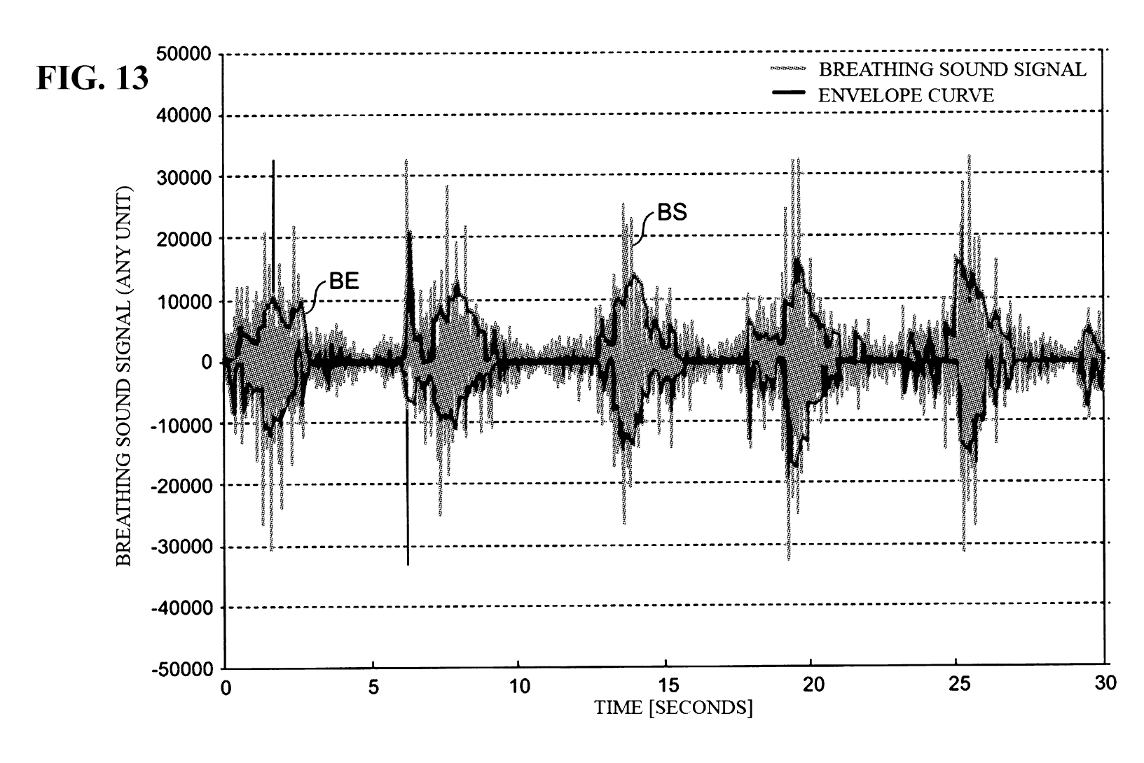

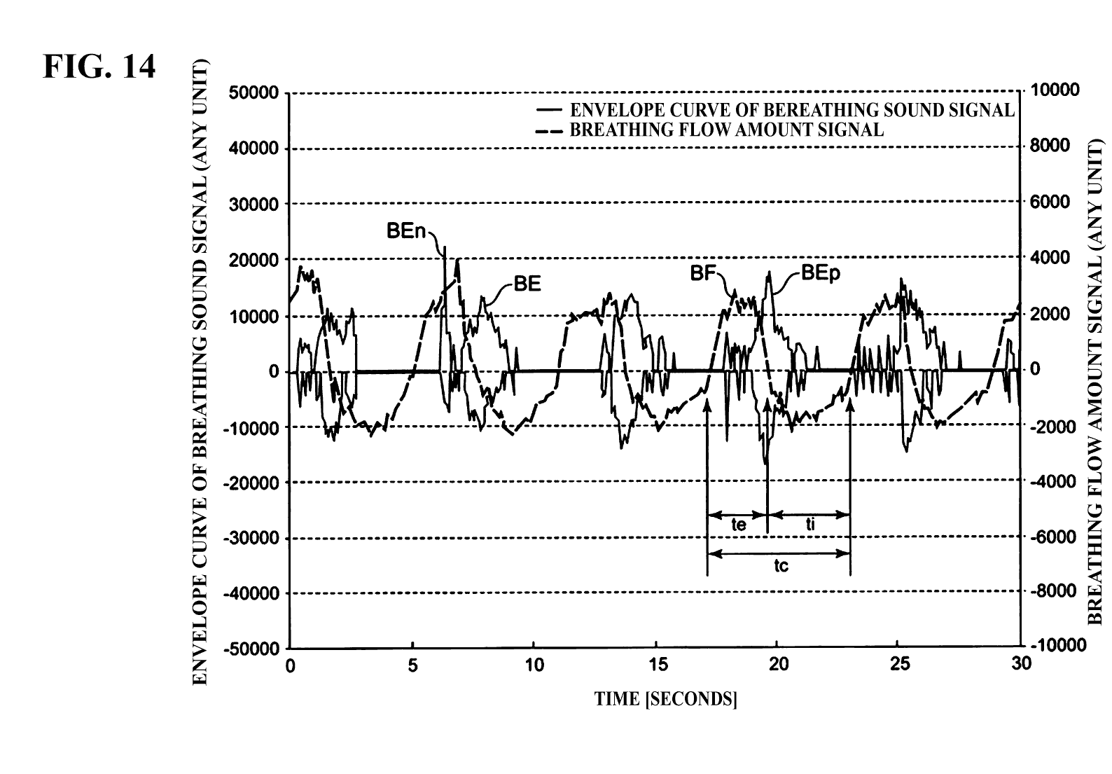

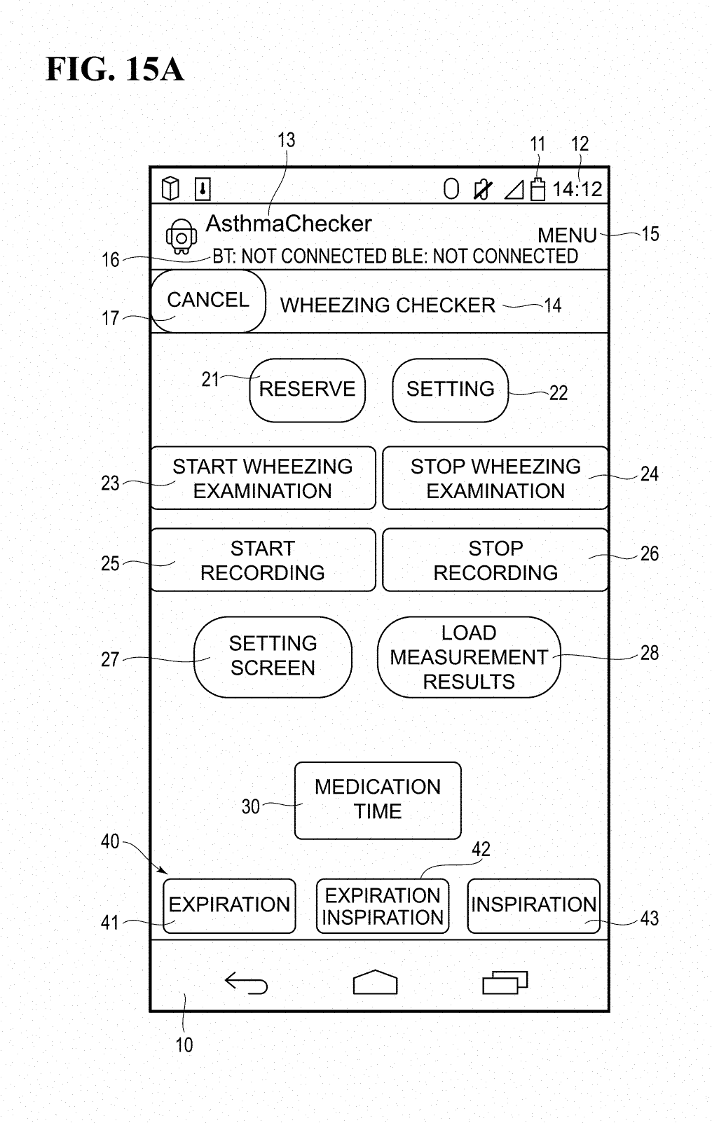

Next, as shown in FIG. 13, the control unit 210 creates an envelope curve BE for the breathing sound signal BS (in this example, if the breathing sound signal BS is 3000 or less, it is replaced with 0 for the sake of ease). If the envelope curve BE for the breathing sound signal BS and the breathing flow amount signal BF shown in FIG. 6 are combined, as shown in FIG. 14, peaks BEp of the envelope curve BE are synchronous with the zero-crossing points at which the breathing flow amount signal BF transitions from positive (inspiration) to negative (expiration). Accordingly, the control unit 210 can distinguish between and identify the expiratory phase te and the inspiratory phase ti in the breathing cycle tc by detecting the peaks BEp of the envelope curve BE. Note that a peak BEn that is misaligned from the cycle of an original peak BEp in the envelope curve BE is ignored as noise based on the average cycle of the original peaks BEp.

Accordingly, upon distinguishing between and identifying the expiratory phase te and the inspiratory phase ti in the breathing cycle tc, the control unit 210 performs recording by storing the phase of the breathing sound signal BS selected using the phase selection switch 40 in the memory 220. Note that the “expiration” display 44 shown in FIG. 16(B) indicates that the expiration phase has been recorded.

Note that in the case of manual recording, when the user 91 presses the “stop recording” switch 26 on the initial menu screen shown in FIG. 15(A), recording performed by the smartphone 200 stops. The wheezing detector 100 receives a signal indicating “stop recording” via the near field wireless communication unit 180 and stops transmission of the breathing sound signal BS. In the automatic recording mode, the period with which the wheezing detector 100 transmits the breathing sound signal BS and the period with which the smartphone 200 performs recording are set to be 30 seconds starting from the recording start time by default (this can be changed and set by the user 91).

In the automatic recording mode, when the wheezing of the infant 90 serving as the measurement subject is relatively large (i.e., when the wheezing is severe), the breathing sound of the infant 90 can be automatically recorded. Accordingly, by replaying the recorded content the next time the infant 90 has a medical examination for example, the user 91 can have a doctor listen to the breathing sound of the infant 90 for when wheezing is severe. As a result, the doctor can more easily diagnose whether or not the infant 90 has asthma and the severity of the asthma, and can easily create a treatment plan. Note that if the smartphone 200 does not receive the alarm signal from the wheezing detector 100 (NO in step S29 of FIG. 19), recording will not be performed.

(6) Thereafter, by pressing the “wheezing sound playback” switch 64 on the screen shown in FIG. 12 for example, the user 91 can play back the breathing sound signal BS stored in the memory 220 with the speaker 260 for example (step S28 in FIG. 19).

According to this operation example, the phase of the breathing sound signal BS selected using the phase selection switch 40 can be recorded. Accordingly, if the phase requested by the doctor during the previous medical examination is selected, for example, the user 91 can have the doctor listen to the recorded content of the phase requested by the doctor in the breathing cycle tc when the user 91 has the doctor listen to the recorded content of the wheezing of the infant 90 during the next medical examination.

Also, the wheezing detection result (the bar graph AT shown in FIGS. 12 and 16) displayed on the display screen 10 of the smartphone 200 may be transmitted to a doctor's computer (a terminal in a hospital) via the network communication unit 290. Accordingly, the user 91 can receive a doctor's diagnosis at a remote location located away from the hospital.

In the above-described embodiment, the wheezing detection apparatus of the present invention is constituted as a wheezing detection system including the wheezing detector 100 and the smartphone 200, but there is no limitation to this. For example, instead of the smartphone 200, it is possible to include a commercially-available computer (a personal computer, etc.). In this case, the above-described “wheezing checker” program is installed in the commercially-available computer.

Also, the wheezing detection apparatus of the present invention may be constituted by only the smartphone 200, for example. In this case, the first microphone 111 and the second microphone 112 are connected to the smartphone 200, and the sound signal processing circuit 115 is mounted in the smartphone 200 (the function of the sound signal processing circuit 115 may be constituted by software and may be executed by the control unit 210). In this case, the wheezing detection apparatus of the present invention can be made small and compact. This configuration is advantageous in the case where the measurement subject is the user of the smartphone 200.

In the above-described embodiment, the temporal change in the frequency of wheezing was displayed as a bar graph AT, but there is no limitation to this. The temporal change in the frequency of wheezing may be displayed as another form of graph. For example, the temporal change in only the percentage (%) of time obtained by combining yellow Y and red R may be displayed as a bar graph.

The above-described embodiment is merely an example, and various modifications are possible without departing from the scope of the invention. The multiple above-described embodiments can be realized independently, but it is also possible to combine embodiments. Also, the various characteristics of the different embodiments can be realized independently, but it is also possible to combine characteristics of different embodiments.

### REFERENCE SIGNS LIST

1 Wheezing detection system

10 Display screen

50 Wheezing detection result display field

100 Wheezing detector

111 First microphone

112 Second microphone

200 Smartphone

AT Bar graph

AT1, AT2, AT3, AT4 Bar

T1, T2, T3, T4 Addition unit period

Tw1, Tw2, Tw3, Tw4 Wheezing period

1-7. (canceled)

8. A wheezing-related information display apparatus comprising:
a breathing sound detection unit configured to detect a breathing sound of a measurement subject and acquire a breathing sound signal in a time series expressing the breathing sound;
a determination processing unit configured to, based on the breathing sound signal, for each predetermined processing unit period, convert the breathing sound signal into a frequency space to acquire a frequency spectrum of the breathing sound, and determine whether or not wheezing is included in the breathing sound based on the area of a dominant peak that has the largest area in a graph of frequency with respect to sound pressure among a plurality of peaks in the frequency spectrum; and
a display processing unit configured to, based on a result of determination performed by the determination processing unit, display a frequency of processing unit periods in which it was determined by the determination processing unit that wheezing is included, in each addition unit period including a plurality of the processing unit periods, as information indicating temporal change in the frequency of the wheezing on a display screen; and
furthermore,
an addition processing unit configured to add up lengths of processing unit periods in which it was determined that the wheezing was included, in the addition periods, and obtain the result as a wheezing period,
wherein the display processing unit displays the information indicating the temporal change in the frequency of the wheezing as a bar graph indicating percentages occupied by the wheezing periods in bars with a certain length that correspond to the addition unit periods.

9. The wheezing-related information display apparatus according to claim 8, wherein
the display processing unit displays a plurality of bars corresponding to the addition unit periods in parallel alignment on the display screen.

10. The wheezing-related information display apparatus according to claim 8, wherein
in each of the processing unit periods, the addition processing unit classifies the power of the wheezing sound into a plurality of levels based on the area of the dominant peak and adds up the lengths of the processing unit periods in which it was determined that the wheezing is included, in each of the classified stages, and
based on the addition performed by the addition processing unit, the display processing unit displays the wheezing periods divided into the levels in the bars corresponding to the addition unit periods.

11. The wheezing-related information display apparatus according to claim 8, further comprising
a medication administration information input unit for inputting information relating to administration of medication to the measurement subject,
wherein the display processing unit displays the information related to the administration of medication for each addition unit period on the display screen, along with the bar graph.

12. The wheezing-related information display apparatus according to claim 8, further comprising:
a phase identification unit configured to identify the breathing cycle of the measurement subject with a distinction made between an expiratory phase and an inspiratory phase, based on the breathing sound signal acquired by the breathing sound detection unit;
a phase instruction input unit configured to input an instruction to select one or both of the expiratory phase and the inspiratory phase of the breathing sound signal; and
a sound recording unit configured to record the phase of the breathing sound signal instructed by the phase instruction input unit.

13. A wheezing-related information display apparatus, comprising:
a breathing sound detection unit configured to detect a breathing sound of a measurement subject and acquire a breathing sound signal in a time series expressing the breathing sound;
a determination processing unit configured to, based on the breathing sound signal, determine whether or not wheezing is included in the breathing sound in each predetermined processing unit period;
a display processing unit configured to, based on a result of determination performed by the determination processing unit, display information indicating temporal change in a frequency of the wheezing on a display screen;
a phase identification unit configured to identify the breathing cycle of the measurement subject with a distinction made between an expiratory phase and an inspiratory phase, based on the breathing sound signal acquired by the breathing sound detection unit;
a phase instruction input unit configured to input an instruction to select one or both of the expiratory phase and the inspiratory phase of the breathing sound signal; and
a sound recording unit configured to record the phase of the breathing sound signal instructed by the phase instruction input unit.
